(19) United States
(12) Patent Application Publication
(10) Pub. No.: US 2007/0150031 A1
(43) Pub. Date: Jun. 28, 2007

(54) OCULAR LIGHT THERAPY DEVICE

(75) Inventor: ANDREW J. BARKER, Calgary, AB (CA)

Correspondence Address: BENNETT JONES;C/O MS ROSEANN CALDWELL, 4500 BANKERS HALL EAST, 855 - 2ND STREET, SW, CALGARY, AB, T2P 4K7, CA

(73) Assignee: THE LITEBOOK COMPANY LTD., Medicine Hat, AB (CA)

(21) Appl. No.: 11/427,692

(22) Filed: Jun. 29, 2006

Related U.S. Application Data
(60) Provisional application No. 60/595,393, filed on Jun. 29, 2005.

(51) Int. Cl. A61N 5/06; A61B 18/18

(52) U.S. Cl. 607/88; 606/4

(57) ABSTRACT

An ocular light therapy device including a housing with a light passage opening, a reflective surface supported by the housing and a light source in the housing, the light source selected to emit light therefrom and positioned to direct the light toward the reflective surface such that light emitted from the light source reflects from the reflective surface through the light passage opening for administration of light therapy.

### FIELD

The present invention relates to ocular light therapy and, in particular, devices and methods for ocular light therapy of light affected conditions.

### BACKGROUND

Light therapy devices are available for treatment of light affected conditions such as, for example, seasonal affective disorder, non-seasonal depression, sleep disorders, shift work adjustment and jet lag. Recently, light therapy devices have been introduced that are sized for convenient and discreet use. In particular, some devices use small light sources that permit ocular light therapy devices to be of such a small size that they are readily transportable. The interest raised by such devices has opened the market for even smaller and more affordable devices.

### SUMMARY

In accordance with a broad aspect of the present invention, there is provided an ocular light therapy device including a light source to produce emitted light and a reflective surface formed as at least a section of a paraboloid of revolution, the reflective surface positioned to reflect light emitted from the light source out of the ocular light therapy device in a substantially collimated reflected beam for ocular light therapy treatment of a user.

In accordance with another broad aspect of the present invention, there is provided ocular light therapy device including a light source and a light-diffusing reflective surface, the light-diffusing reflective surface positioned to reflect light emitted from the light source out of the ocular light therapy device in a form diffused 5° to 30°.

In accordance with another broad aspect of the present invention, there is provided an ocular light therapy device including a housing, a reflective surface, a high-power LED selected to emit light therefrom, the high-power LED positioned to emit light toward the reflective surface such that reflected light from the high-power LED is passed from the housing.

In accordance with another broad aspect of the present invention, there is provided ocular light therapy device including a housing with a light passage opening, a reflective surface supported by the housing and a light source in the housing, the light source selected to emit light therefrom and positioned to direct the light toward the reflective surface such that light emitted from the light source reflects from the reflective surface through the light passage opening for administration of light therapy.

In accordance with other broad aspects of the present invention, methods are provided for ocular light therapy using any of the devices disclosed herein.

It is to be understood that other aspects of the present invention will become readily apparent to those skilled in the art from the following detailed description, wherein various embodiments of the invention are shown and described by way of illustration. As will be realized, the invention is capable for other and different embodiments and its several details are capable of modification in various other respects, all without departing from the spirit and scope of the present invention. Accordingly the drawings and detailed description are to be regarded as illustrative in nature and not as restrictive.

### BRIEF DESCRIPTION OF THE DRAWINGS

Referring to the drawings wherein like reference numerals indicate similar parts throughout the several views, several aspects of the present invention are illustrated by way of example, and not by way of limitation, in detail in the figures, wherein:

FIG. 1 is a schematic sectional view of one ocular light therapy device according to the present invention, the device being in the closed position.

FIG. 2 is a schematic sectional view of the light therapy device of FIG. 1 in the open position, for use.

FIG. 3 is a top perspective view of a light therapy device according to another embodiment and in the closed position.

FIG. 4 is a top perspective view of the light therapy device of FIG. 3 in the open position for use.

### DETAILED DESCRIPTION OF VARIOUS EMBODIMENTS

The detailed description set forth below in connection with the appended drawings is intended as a description of various embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details.

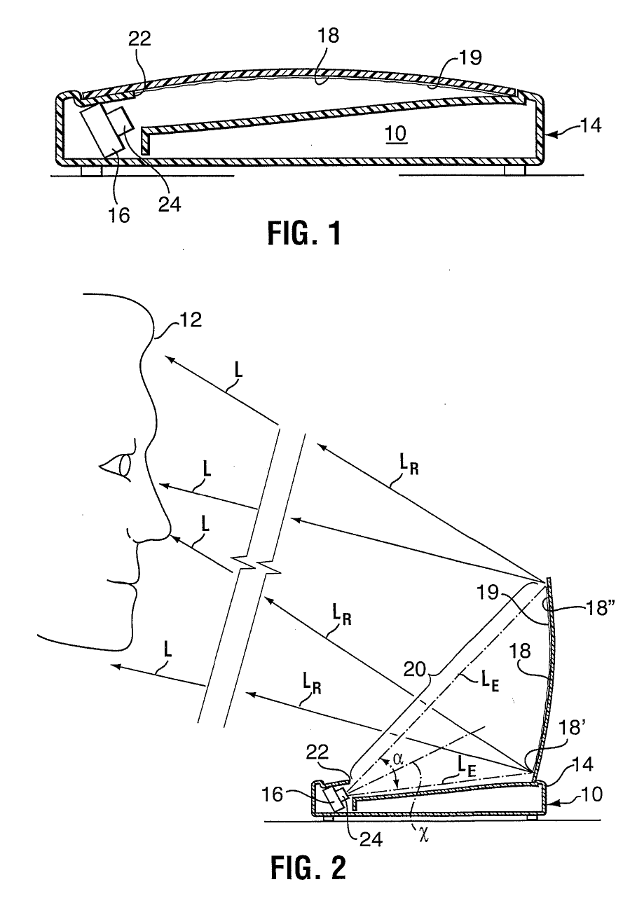

With reference to FIGS. 1 and 2, there is shown an ocular light therapy device 10 for use to treat a user 12 with a light affected condition. The device generates light L_{R} to be shone into the eyes of user 12. Such ocular light therapy has been shown to alleviate at least some light affected disorders.

Ocular light therapy device 10 includes a housing 14, a light source 16 and a reflective surface 18.

The housing may serve to support and protect light source 16 and reflective surface 18 and the various mechanisms to power and control the light source and device generally. Housing 14 may include a light passage opening, generally indicated at 20, that permits light from the light source to pass from the housing to a user. The light passage opening can take various forms. In the illustrated embodiment, light passage opening 20 is defined by the opening between housing edges 22 and reflective surface, the opening being formed when reflective surface 18 is in the opened position.

Housing 14 may be formed of various materials and through various processes, as will be appreciated. In order to enhance portability, the housing may be formed to be very small, for example of a size capable of being hand-held. With such a size, the device may be easily placed in a handbag or briefcase. Housing 14 may be formed of durable materials, such as may include plastics, metals, etc.

Light source 16 may take various forms. Light source may be one or more light emitting devices using various technologies such as incandescent, fluorescent including cold cathode fluorescent, halogen, light emitting diode (LED) including organic LED (OLED), high intensity LED, fiber optics, etc. The reflected light must be capable of offering ocular light therapy and this may require consideration for the selection of the light source.

In one embodiment, a small sized and durable light source may be useful. LED-based light sources have proven to be quite durable and of a small size. Thus, in one embodiment, light source 16 may include one or more LED light emitting devices. In one embodiment, light source 16 may include one or more high-power LEDs. The main difference between a high power LED and a standard LED lies in the internal design wherein a high power LED exhibits greatly improved heat transfer characteristics, permitting higher current operation, with a larger light emitting surface when compared to a standard LED. High power LEDs tend to offer better maintenance of light output over time. A high power LED includes a heat sink slug in heat transfer communication with the light emitting surface, which a standard LED does not have. Also, high power LEDs tend not to have a dual bottom pin configuration, instead including a side protruding lead.

Since high-power LEDs may generate more lumens per watt than a traditional LED, the use of high-power LEDs may permit the size, weight and cost of the light source to be reduced over a light source of similar light output using other kinds of light emitting devices. High-power LEDs are available, for example from Lumileds Lighting US, LLC. (eg. Luxeon™ products) and from Nichia Corporation (eg. Jupiter™ products). High-power LED's capable of emitting 40 to 120 lumens may be used. In one embodiment, a high-power LED of 40 to 50 lumens may be used.

Although a device using one high-power LED may offer the most simple and cost effective solution to a source of light, higher output products, as for example may be desired for use under medical supervision, may incorporate more than one high-powered LED. Where more than one light source is used, some or all of the various light sources may be aimed at reflective surface 18 such that their emitted light is delivered to a user by reflection. In one embodiment, wherein more than one light source is used to increase overall light output, the individual sources may be aimed in such a way that their output beams overlap on a common area of reflective surface 18. The area of reflective surface 18 may optionally be increased in order to reduce perceived glare or to improve ocular safety.

Alternatively, higher output may be achieved by assembling an array of individual reflective surfaces 18, each surface being arranged to receive the output beam from one or more LEDs and redirect that output onto the face of a user.

The light source may be white or peaked in any particular wavelength such that light of any of various colors may be emitted. Where more than one light source is used, the various light sources may be selected to be of differing wavelength outputs such that the combined emitted light has a specifically tailored overall spectrum of emitted light. If desired, the device may include an intensity selector so that the intensity of the emitted light from the light source may be selected such that the output of light is sufficient and suitable for ocular light therapy.

Light source 16 may be supported by the housing such that light emitted therefrom is directed toward reflective surface 18 such that reflected light from the light source is passed from the housing for ocular light treatment. For example, light source 16 may be positioned to direct its light L_{E} toward the reflective surface such that light emitted from the light source reflects from the reflective surface before being passed through light passage opening 20. In one embodiment, light source 16 may be positioned such that light emitted therefrom is aimed toward reflective surface 18. In another embodiment, refraction or reflection may be used to collect and/or direct light from the light source to the reflective surface. Refraction and reflection may also be used to control the light beam directed at reflective surface 18 so that light from the light source is either (i) captured and focused for efficient use in light therapy, rather than being lost laterally as may occur by spill over beyond the edges of the reflective surfaces, or (ii) diffused to create a light beam more suitable for delivery for ocular light therapy. In one embodiment, it may be desirable to select the device set up and/or components such that the light emitted from the light source is effectively and efficiently captured and reflected to provide light therapy. For example, in the illustrated embodiment, the light source is mounted in the housing such that its center axis χ of light emission is directed towards reflective surface and a lens 24 is mounted between light source 16 and reflective surface 18 to refract, and, thereby, collect and direct, light from the light source to the reflective surface. In one embodiment, for example, a lens may be used that focuses light from the light source to create a beam of light spread at an angle a selected to substantially fill the surface area of the reflective surface. In some embodiments, it may be useful to select the size of the reflective surface that is desired to be used and then work back with consideration as to the light source to determine whether there is a need to focus the light emitted from the light source. For example, the size of reflective surface 18 is dictated by product aesthetics for portability (smaller is better) and by usage factors such as glare reduction and ocular safety (larger is better). Once the design compromise on size is established, the output beam angle from the source is tailored via an intermediate reflective or refractive optical component to efficiently fill the reflective surface with light emanating from the source at its chosen location. In the illustrated embodiment, a small diameter light source (less than two inches in diameter) is used and a reflective surface of less than about 6×6 inches was considered of interest and the angle a is less than 90° and in one embodiment 30° to 60° relative to the center axis χ of the light source.

When using a high intensity light source a reflected and/or diffused light may be most safe for delivery to a user. In the illustrated embodiments, for example, only reflected light is delivered through the opening from the device toward a user.

Reflective surface 18 acts to reflect and direct light from the light source such that it is passed from the device to a user as reflected light L_{R} in a defined beam to create a patch or window of light at a therapy distance. Light reflection may be provided by use of surfaces of aluminum, silver or other materials, in the form of paint, powder, foil, etc.

In one embodiment, reflective surface 18 is selected to diffuse the light reflected therefrom such that the reflected light creates a visual impression of light emerging from a large area source of substantially uniform, moderate intensity rather than from one or more intensely bright localized light sources. For example, following diffusion at the reflective surface light from the single light sources may be overlapped on the face of a user positioned at a normal therapy distance, for example of 1 to 3 feet from the device. At the same time, it may be desired that the reflective surface direct the light along a relatively narrow path such that the light is concentrated efficiently to only illuminate a therapy window of at least a size to cover a user's eye and generally no larger than shoulder width at the therapy distance. Reasonably, this window may be considered as 8 to 24 inches wide at 1 to 3 feet from the device. The reflective surface may diverge each incident light ray into a cone of full angle to deliver such a patch of light. In one embodiment, for example, a 6×6 inch reflective surface may be considered of interest and diffusion of light rays passing from the reflective surface out of the device may be selected to be approximately 5° to 30° or possibly even approximately 5° to 15° to create a patch of light of 10 to 18 inches wide at a distance of 18 to 30 inches from the device.

Reflective surface 18 may be curved to reflect and diffuse or concentrate the light. For example, the reflective surface may be curved convexly or concavely (as shown) over all or a portion of its surface area. It may be useful to select the shape of the curvature of surface 18 to efficiently capture emitted light and direct it along a selected path toward a user.

The curvature may, for example, be selected to be concave such as may include circular or parabolic cylindrical forms or those having curvature about two orthogonal axes such as those being semi-spherical or defining a section of a paraboloid of revolution. In one embodiment, reflective surface 18 may have paraboloidal curvature wherein the radius of curvature of the reflective surface varies from its lower end 18′ to its upper end 18″, with the reflective surface having a shorter radius (being more curved) at the lower end than at the upper end. The curvature of such a reflector may be defined by a section of a paraboloid of revolution (a parabola rotated about its axis), so that the reflective surface may have surface curvature about two orthogonal axes including from upper end to lower end and from side to side. A paraboloidal curvature offers a reflective surface that may efficiently capture emitted light from a light source positioned close to its focal point, and reflect it as a substantially parallel, collimated beam of reflected light L_{R}.

Reflective surface 18 may be a true mirror (i.e. perfectly smooth). If reflected light L_{R} is desired to be passed in a diffused state to a user, (i) a true mirror may be used with a separate light diffusing material positioned to act on the light before or after impinging the reflective surface (ii) a true mirror may be used with diffusing material applied thereto or (iii) a non-true reflective surface may be used.

To create a diffusing effect on reflective surface 18, it will be appreciated that a mirror surface can be textured, as by sand blasting, brushing, scoring, coating, etc. This may create a light-diffusing effect. However, the random nature of some surface texturing may create an uncontrolled degree of diffusion. It may, therefore, be desirable to select a form of light-diffusion so that the actual degree of diffusion may be substantially controlled to create a specific effect.

In one embodiment, for example, reflective surface 18 may include a regular or random array of curved reflector elements. The individual reflector elements may be curved convexly or concavely. For example, each reflector may be 0.5 to 2.0 mm in diameter and positioned to form in whole or in part the reflective surface. In another embodiment, the reflective surface may include a mirrored surface applied on a substrate having regular or irregular surface undulations incorporating surface slope changes.

In yet another embodiment, reflective surface 18 may be formed by use of a light-diffusing material 19 positioned in front of (in contact with or spaced from) a mirror surface. Light diffusing materials may include refractive transparent or semi-transparent materials including uniform or varying surface structures or thickness. Such materials may in one embodiment be in contact with the reflective surface, as by application over surface 18 or by forming surface 18 on the diffusing material. In such a reflective surface, the light-diffusing material may be selected to interact with the light rays twice: when entering the coating before impinging on the mirror surface and when passing again through the coating after being reflected from the mirror surface. In one such embodiment, a light-diffusing coating may be used that diverges light 5° to 15° at each pass.

Of course, a protective coating, for example of dielectric, can be applied over reflective surface 18 or diffusing material, if desired.

Although a device with only one reflective surface is shown, more than one reflective surface can be used if desired. If more than one surface is used, the plurality of surfaces can be positioned in side-by-side relation and/or can be vertically stacked.

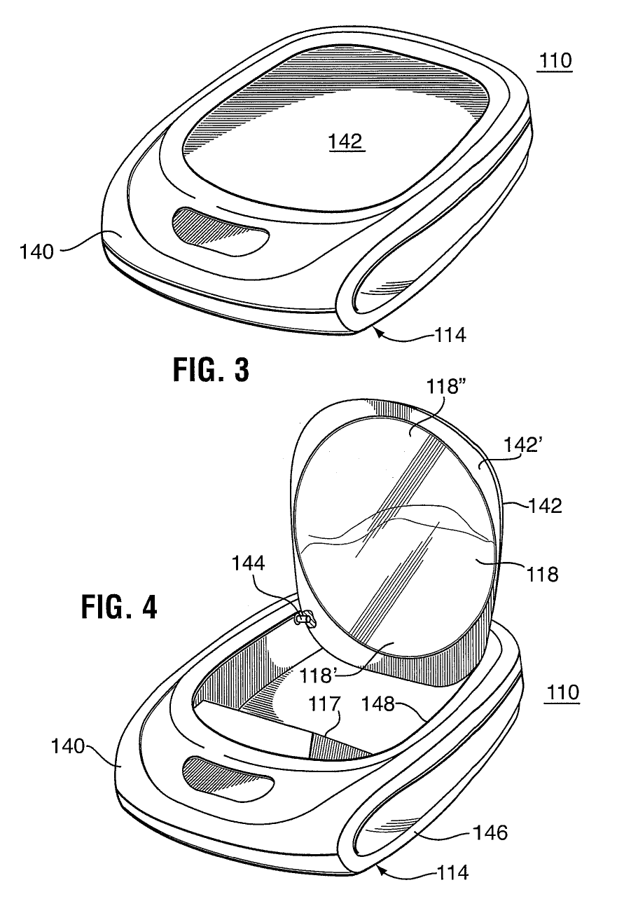

With reference to FIGS. 3 and 4, another ocular light therapy device 110 is shown. Ocular light therapy device 110 includes a housing 114, a light source in a mounting support 117 and a reflective surface 118.

Housing 114 may be sized to be hand held such as approximately 4 to 8 inches in length and width and 1 to 2 inches thick. Housing 114 in the illustrated embodiment is approximately 6×6×1.25 inches. Housing 114, as shown in the illustrated embodiment, may include a base 140 and a lid 142 pivotally connected by a hinge 144 to base 140. Base 140 may include a lower surface 146 formed, as by defining a flat surface, legs, a mounting structure, a support arm, etc., to support the device in a therapy position on a support and an upper surface with an opening 148 therein. Lid 142 is pivotally connected relative to opening 148 such that the lid can be moved from a closed position wherein it extends over and covers the opening to an open position wherein opening 148 is at least in part exposed. In the closed position, the inner facing portion 142′ of the lid faces toward opening 148.

Base 140 supports the light source, which in the illustrated embodiment is a high-powered LED, such as one available from the Luxeon™ product line capable of emitting light in the order of about 100 lumens with a light emitting opening of about 3 to 4 mm. A lens (not shown) may be used to focus the LED from its 180° light emission range to approximately 40° to 60° from the center axis of the light.

Lid 142 on its inner facing surface supports reflective surface 118. Reflective surface 118 is curved to define a section of a paraboloid of revolution. The parabolic curvature causes lower end 118′ to be more curved than upper end 118″. A user looking into such a parabolic reflective surface may see an intense, magnified image of the front of the LED optic and the residual divergence in the collimated beam may be too small to create a patch of light of adequate width on a user's face. By selecting a large diameter optic for positioning over the LED and a suitable sized surface 118, a sufficiently large patch of light may be reflected onto the user at a therapy distance. This arrangement of light source, optic and reflective surface may be very efficient at using light from the light source and creating a patch of light on the users face with sharply defined edges. However, in the illustrated embodiment, reflective surface 118 is further textured to diffuse light reflected therefrom. By addition of a diffusing characteristic to the parabolic reflective surface, the overall divergence of the beam leaving the diffusing reflector can be achieved to create a patch of light of 8 to 24 inches in width at a therapy distance. With the diffusing characteristic on the parabolic surface, a user now sees the curved reflector as a secondary source of light, uniformly bright across its surface provided that the beam of light emanating from the LED optic is uniform.

In the illustrated embodiment, for example, reflective surface 118 includes a mirror surface and a layer thereover defining randomized surface relief structures. The surface relief structures are substantially transparent to light of various wavelengths and permit controllable angular distribution such that light passed therethrough becomes diffused. Such surface relief structures are available, for example, under the tradename LSD® (Light Shaping Diffusers available from POC. Such products may, for example, be holographically recorded and fully randomized (non-periodic) structures applied over or incorporated with light reflective materials. The surface relief structures may provide controlled angular light divergence, emulating a negative lens. In the illustrated embodiment, a surface treatment may be selected such that reflected light is diffused by 5° to 15°.

Lid 142 and the light source are positioned such that reflective surface 118 receives light from the light source, when the lid is opened. Reflective surface 118 then reflects, and thereby directs the light, out of the device through light passage opening, defined between the lid and the base, in the open position. Selection of the reflective surface's properties of curvature and diffusion can ensure that the light reflected from reflective surface 118 passes along a relatively well-defined illumination path creating a “window” of light for use by a user. In use, the device can be placed on a table or other support surface, and the lid can be raised to direct the light “window” where needed. The tilt angle of the lid can be adjusted, as by use of an adjustable hinge 146, to accommodate variations in subject eye height relative to the device.

In one embodiment, device 110 can be controlled by a switch that operates to turn on and off the LED source upon opening and closing the lid. In another embodiment, the light source may be powered automatically, for example with a soft start following opening of the lid, and go off after a selected time period (for example 20-30 mins) or upon closing the lid. The device may include a memory function for example for programming by a doctor or for monitoring compliance. The device may be powered by a cord (i.e. a standard AC supply or USB) and/or through battery power.

If necessary, more than one LED could be used with one or more reflective surfaces. If more then one high power LED is used with a single reflective surface, it may be useful to mount the LEDs in side-by-side relation horizontally (during use). Alternately or in addition, several LED/reflective surface modules could be positioned or connected alongside each other. While manufacturing cost, portability and visual appearance favor a single reflective surface system, of course, devices for use under medical supervision may incorporate more complex arrangements.

Ocular light therapy devices may include other features such as, for example, displays, indicator lights, audio systems, timers and compliance monitoring software, as desired.

The previous description of the disclosed embodiments is provided to enable any person skilled in the art to make or use the present invention. Various modifications to those embodiments will be readily apparent to those skilled in the art, and the generic principles defined herein may be applied to other embodiments without departing from the spirit or scope of the invention. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims. No claim element is to be construed under the provisions of 35 USC 112, sixth paragraph, unless the element is expressly recited using the phrase “means for” or “step for”.

I claim:

1. An ocular light therapy device including a light source to produce emitted light and a reflective surface formed as at least a section of a paraboloid of revolution, the reflective surface positioned to reflect light emitted from the light source out of the ocular light therapy device in a substantially collimated reflected beam for ocular light therapy treatment of a user.

2. The ocular light therapy device of claim 1 further comprising an optic positioned between the light source and the reflective surface.

3. The ocular light therapy device of claim 1 further comprising a light-diffusing material positioned to act on the light emitted by the light source at least one of (i) before reflection by the reflective surface and (ii) after reflection by the reflective surface.

4. An ocular light therapy device including a light source and a light-diffusing reflective surface, the light-diffusing reflective surface positioned to reflect light emitted from the light source out of the ocular light therapy device in a form diffused 5° to 30°.

5. The ocular light therapy device of claim 4 further comprising an optic positioned between the light source and the light-diffusing reflective surface.

6. The ocular light therapy device of claim 5 wherein the optic focuses light from the light source to the light-diffusing reflective surface.

7. The ocular light therapy device of claim 4 wherein the light diffusing reflective surface includes a mirrored surface and a material layer positioned between the mirrored surface and the light source defining randomized surface relief structures.

8. The ocular light therapy device of claim 4 wherein the light diffusing reflective surface includes a non-true mirror.

9. The ocular light therapy device of claim 8 wherein the non-true mirror is provided by surface texturing.

10. The ocular light therapy device of claim 4 wherein the light-diffusing reflective surface is curved about two orthogonal axes.

11. An ocular light therapy device including a housing, a reflective surface, a high-power LED selected to emit light therefrom, the high-power LED positioned to emit light toward the reflective surface such that reflected light from the high-power LED is passed from the housing.

12. The ocular light therapy device of claim 11 further comprising an optic positioned between the high-power LED and the reflective surface.

13. The ocular light therapy device of claim 12 wherein the optic focuses light from the high-power LED to illuminate the reflective surface substantially without lateral loss of light from the high-power LED beyond the reflective surface without reflection therefrom.

14. The ocular light therapy device of claim 11 wherein the light-diffusing reflective surface is curved about two orthogonal axes.

15. The ocular light therapy device of claim 11 wherein the housing includes a base and a second member, the high-power LED being mounted in the base and the reflective surface being mounted on the second member.

16. The ocular light therapy device of claim 15 wherein the second member forms a lid over the high-power LED and is moveable to a position for operation of the device to reflect light from the high-power LED out of the housing.

17. An ocular light therapy device including a housing with a light passage opening, a reflective surface supported by the housing and a light source in the housing, the light source selected to emit light therefrom and positioned to direct the light toward the reflective surface such that light emitted from the light source reflects from the reflective surface through the light passage opening for administration of light therapy.

18. The ocular light therapy device of claim 17 wherein the light passage opening is defined as an opening between the light source and the reflective surface.

19. The ocular light therapy device of claim 17 wherein the reflective surface forms a lid over the light source, the reflective surface being moveable between a position operating as a lid and a position for operation of the device to reflect light from the light source out of the housing.

20. The ocular light therapy device of claim 17 wherein the reflective surface is pivotally connected to a base portion of the housing.

21. A method for ocular light therapy using a device according to any of the foregoing claims to direct light to a user positioned a therapy distance from the device.

22. The method of claim 21 wherein the reflective surface directs a patch of light of about shoulder width to the user.

23. The method of claim 21 wherein the reflective surface is oriented to direct a patch of light into the eyes of a user.

24. The method of claim 21 wherein the device is positioned on a support surface.
